(19) United States
(12) Patent Application Publication
(10) Pub. No.: US 2005/0175677 A1
(43) Pub. Date: Aug. 11, 2005

(54) Lotion applicator

(76) Inventors: Nina L. Walters, Farmington Hills, MI (US); Vicki Anne West, Bloomfield Hills, MI (US)

Correspondence Address: Michael D. Wiggins, 950 Harmon, Birmingham, MI, 48009, US

(21) Appl. No.: 10/774,968

(22) Filed: Feb. 9, 2004

(51) Int. Cl. A46B 5/02; A61K 9/70

(52) U.S. Cl. 424/443; 401/6

(57) ABSTRACT

A lotion applicator for applying lotion to a body of a person includes an applicator body and a handle that extends from the applicator body. A sheet is impregnated with lotion. A fastener is associated with at least one of the lotion-impregnated sheet and the applicator body and removably attaches the lotion-impregnated sheet to the applicator body. The lotion that is impregnated in the lotion-impregnated sheet is at least one of bug spray, sunburn lotion, dry skin lotion, sunscreen, tanning lotion, pain reliever, muscle relaxing lotion, acne medication, anti-itch lotion, self-tanning lotion, hair removal lotion, body hair bleaching agents, perfume, and topical medications.

The present invention relates to lotion applicators, and more particularly to a lotion applicator with replaceable lotion-impregnated sheets.

### BACKGROUND OF THE INVENTION

Oftentimes, a consumer needs to apply lotion on his or her body. For example, a sunbather applies lotion such as sunscreen and/or moisturizer to his or her body while sunbathing. It may be difficult for the sunbather to apply the lotion to certain portions of his or her body without help. For example, most people have a difficult time reaching their lower and/or mid-back region. Other sunbathers may have problems reaching their feet and/or lower legs. Elderly people may have difficulty reaching additional areas of their bodies.

The failure to apply lotion to all of the exposed portions of the sunbather's body may lead to sunburn. When the sunbather returns home, he or she may also need to apply sunburn lotion or other skin care products such as moisturizer. It may be difficult for the sunbather to apply the sunburn lotion to the sunburned areas to obtain relief.

There are a wide variety of other topical lotions that may be difficult to apply properly without assistance. Other exemplary topical lotions may include (but are not limited to) bug spray, sunburn lotion, dry skin lotion, sunscreen, tanning lotion, pain relieving lotion, muscle relaxing lotion, acne medication, anti-itch lotion, self-tanning lotion, hair removal lotion, body hair bleaching agents, perfume, and topical medications.

Lotion applicators are sometimes used to apply lotion to areas of the body that are difficult to reach. One conventional lotion applicator includes an applicator body with an internal cavity and a permeable surface such as a sponge that is in fluid communication with the cavity. A handle extends from and/or is integrated with the applicator body. In use, a user fills the internal cavity of the lotion applicator with lotion. The permeable surface absorbs and/or wicks the lotion to an outer surface thereof. Using the handle, the user moves the permeable surface of the applicator along areas that are to be treated.

When the user wants to use a different type of lotion, however, the user must empty the cavity and clean the permeable surface. Even when the lotion remains the same, the lotion absorbed by the permeable surface may tend to dry and/or the permeable surface may begin to emit odors, become worn and/or grow mold, fungus, or bacteria.

### SUMMARY OF THE INVENTION

A lotion applicator according to the present invention is used to apply lotion to a body of a person. The lotion applicator includes an applicator body and a handle that extends from the applicator body. A sheet is impregnated with lotion. A fastener that is associated with the applicator body and/or the sheet removably attaches the lotion-impregnated sheet to the applicator body.

In other features, the lotion in the lotion-impregnated sheet is at least one of bug spray, sunburn lotion, dry skin lotion, sunscreen, tanning lotion, pain relieving lotion, muscle relaxing lotion, acne medication, anti-itch lotion, self-tanning lotion, hair removal lotion, body hair bleaching agents, perfume, and topical medications.

In still other features, the fastener includes a flexible member that is located adjacent to a hole in the applicator body and that includes at least two radially inwardly projecting flexible members that releasably engage the lotion impregnated sheet.

In still other features, the handle and the applicator body are integrated. Alternatively, the handle includes a first connector and the applicator body includes a second connector. The first connector mates with the second connector to attach the handle to the applicator body.

In other features, the applicator body includes a top surface having an arcuate cross section and a substantially planar bottom surface.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIGS. 1A and 1B are cross sectional views of exemplary hollow and solid applicator bodies according to the present invention;

FIG. 2 is a plan view of a bottom surface of the applicator body of FIGS. 1A and 1B;

FIG. 3 is a plan view of a top surface of the applicator body of FIGS. 1A and 1B;

FIGS. 4A, 4B and 4C are plan views of applicator handles having varying lengths;

FIG. 5 is a plan view illustrating the applicator handle, the applicator body and a first exemplary lotion-impregnated sheet; and

FIG. 6 is a plan view illustrating the applicator handle, the applicator body and a second exemplary lotion-impregnated sheet.

### DETAILED DESCRIPTION OF THE PREFERRED EMBODIMENTS

The following description of the preferred embodiment(s) is merely exemplary in nature and is in no way intended to limit the invention, its application, or uses. For purposes of clarity, the same reference numbers will be used in the drawings to identify similar elements.

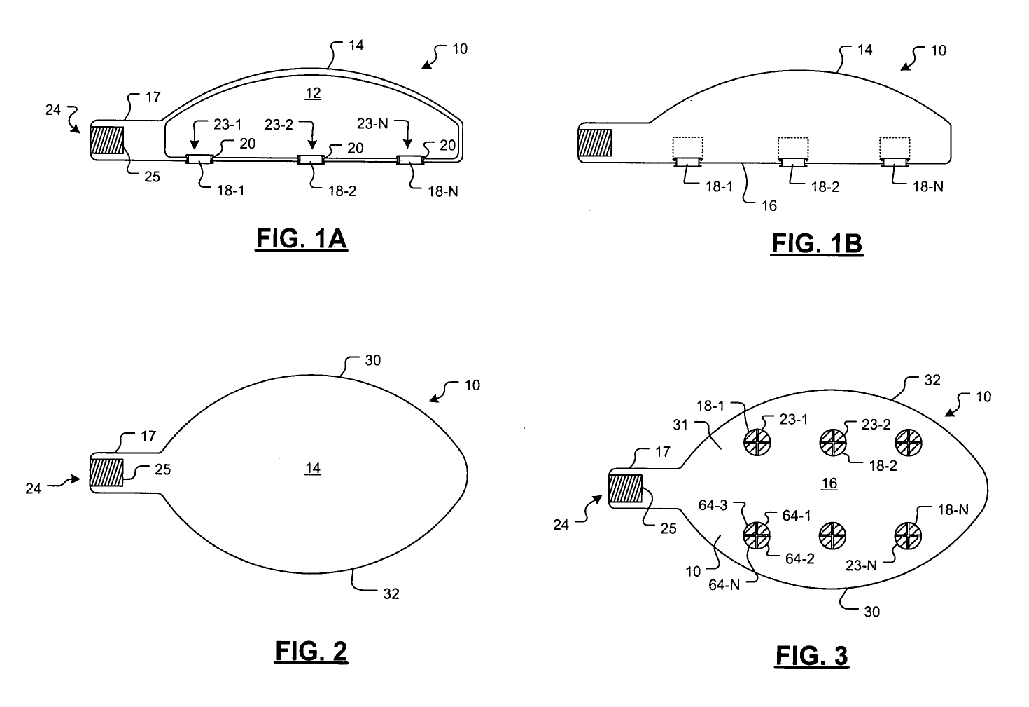

Referring now to FIGS. 1A and 1B, cross sectional views of a hollow and solid, respectively, applicator bodies 10 according to the present invention are shown. In FIG. 1A, the applicator body 10 forms a cavity 12 and includes a top surface 14, a bottom surface 16, and an end portion 17. In FIG. 1B, the applicator body 10 is solid. Skilled artisans will appreciate that the applicator body 10 may be partially solid, ribbed, and/or any other suitable configuration. One or more fasteners 18-1, . . . , 18-N (collectively identified as fasteners 18) that are associated with the applicator body 10 are used to retain lotion-impregnated sheets on the applicator body 10 as will be described below.

In one implementation, the fasteners 18 include a hole 20 that is formed in the bottom surface 16. While circular holes are shown (for example, in FIG. 3), the holes 20 can have any suitable shape. As will be described below, the fastener 18 includes sheet retainers 23-1, 23-2, . . . , and 23-N (collectively identified as sheet retainers 23) (shown and described in FIG. 3 below) that are located in the hole 20. A first connector 24 is located in the end portion 17 of the applicator body 10. The first connector 24 connects the applicator body 10 to a handle that will be described below. In one implementation, the first mating connector 24 is a threaded female bore 25 that is formed at one end of the applicator body 10. As can be appreciated, the handle may also be formed integrally with the applicator body 10.

Referring now to FIGS. 1, 2 and 3, opposite arcuate sides 30 and 32 of the applicator body 10 extend outwardly from a central portion 31 of the applicator body 10. While a particular shape has been shown in FIGS. 1-3 for the applicator body 10, any suitable shape can be used. For example, the shape of the applicator body 10 can be tailored for a particular use. In one implementation, the applicator body has a shape that is similar to a bar of soap.

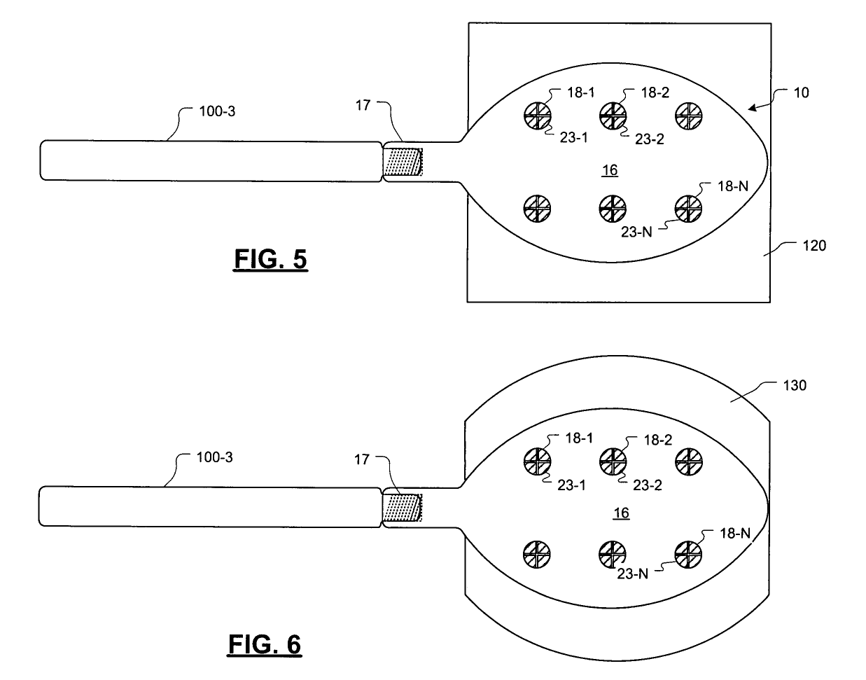

In one implementation, the sheet retainer 23 includes one or more flexible projecting portions 64-1, . . . , and 64-M (collectively 64) that project radially inwardly towards a center of the hole 20. In one implementation, the sheet retainers 23 are made of rubber. In use, lotion-impregnated sheets (described in FIGS. 5 and 6) are wrapped around the applicator body 10. Opposite sides of the sheets are inserted into the holes 20 and releasably engaged by the sheet retainers 23.

Skilled artisans will appreciate that any type of fastener may be employed including but not limited to Velcroe fasteners, hook fasteners that hook the lotion-impregnated sheets, snapping fasteners, and/or spring-based fasteners.

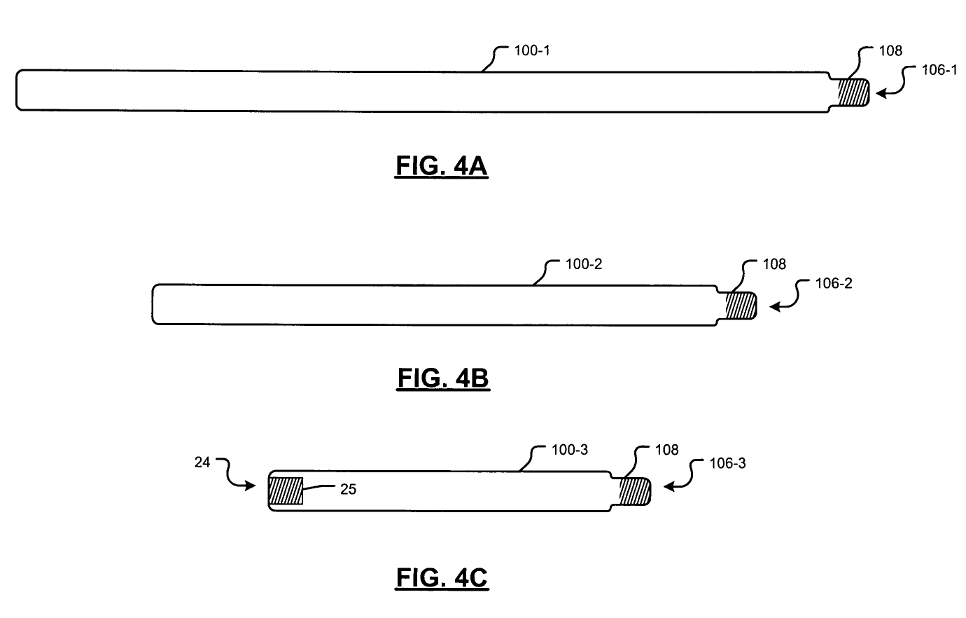

FIGS. 4A, 4B and 4C are cross sectional views of applicator handles 100-1, 100-2, and 100-3, respectively (collectively handles 100), having varying lengths. The applicator handles 100 include second connectors 106-1, 106-2, and 106-3, respectively (collectively second connectors 106), that are matingly received by the first connector 24. While threaded connectors are shown, any conventional fastener may be used. The particular length of the handle 100 that is selected by the user may depend upon the particular application. As can be seen in FIG. 4C, the handle 100-3 includes the first connector 24 that is located at an opposite end to allow another handle 100 to be connected to further extend the reach.

Referring now to FIG. 5, the applicator handle 100, the applicator body 10, the fasteners 18 and a first exemplary lotion-impregnated sheet 120 are shown. In use, the user wraps the applicator body 10 with the sheet 120 and uses the fasteners 18 to releasably attach the lotion-impregnated sheet 120 to the applicator body 10. When the lotion on the sheet 120 is exhausted, the sheet 120 becomes dirty, and/or the user needs to use a different type of lotion, the sheet 120 is removed and discarded. A new lotion-impregnated sheet 120 is attached for further use.

In FIG. 5, the lotion-impregnated sheet 120 has a generally rectangular cross section. However, skilled artisans will appreciate that the lotion-impregnated sheet 120 can have a variety of other shapes. For example, FIG. 6 illustrates one exemplary alternate lotion-impregnated sheet 130. However, it is readily apparent that the lotion-impregnated sheets can have any suitable shape. The lotion-impregnated sheets 120 are preferably made of a soft, absorbent material. For example, the material may be similar to disposable hand or face wipes.

While the sheets are described as “lotion-impregnated” sheets, the term lotion is mean to include any material that is to be applied to a user's body using the lotion applicator. For example, the term lotion includes, but is not limited to, bug spray, sunburn lotion, dry skin lotion, sunscreen, tanning lotion, pain relieving lotion, muscle relaxing lotion, acne medication, anti-itch lotion, self-tanning lotion, hair removal lotion, body hair bleaching agents, perfume, topical medications, and/or any other material that is generally applied to the body.

Based on the foregoing disclosure, it will be apparent that the lotion applicator according to the present invention allows a user to apply any type of lotion to difficult to reach parts of his or her body. It will further be appreciated that the type of lotion that is applied can be readily changed by discarding the current sheet and replacing it with a sheet having the desired lotion.

Those skilled in the art can now appreciate from the foregoing description that the broad teachings of the present invention can be implemented in a variety of forms. Therefore, while this invention has been described in connection with particular examples thereof, the true scope of the invention should not be so limited since other modifications will become apparent to the skilled practitioner upon a study of the drawings, the specification and the following claims.

1. A lotion applicator for applying lotion to a body of a person, comprising:
an applicator body;
a handle that extends from said applicator body;
a sheet that is impregnated with lotion; and
a fastener that is associated with at least one of said lotion-impregnated sheet and said applicator body and that removably attaches said lotion-impregnated sheet to said applicator body.

2. The lotion applicator of claim 1 wherein said lotion that is impregnated in said lotion-impregnated sheet is at least one of bug spray, sunburn lotion, dry skin lotion, sunscreen, tanning lotion, pain relieving lotion, muscle relaxing lotion, acne medication, anti-itch lotion, self-tanning lotion, hair removal lotion, body hair bleaching agents, perfume, and topical medications.

3. The lotion applicator of claim 1 wherein said fastener includes a flexible member that is received in a hole in said applicator body and that includes at least two radially inwardly projecting, flexible members that engage said lotion-impregnated sheet.

4. The lotion applicator of claim 1 wherein said handle and said applicator body are integrated.

5. The lotion applicator of claim 1 wherein said handle includes a first connector and said applicator body includes an end portion with a second connector and wherein said first connector mates with said second connector to attach said handle to said applicator body.

6. The lotion applicator of claim 1 wherein said applicator body includes a top surface having an arcuate cross section and a bottom surface having a substantially planar surface.

7. A lotion applicator for applying lotion to a body of a person, comprising:
an applicator body;
a handle that extends from said applicator body;
a sheet that is impregnated with lotion, wherein said lotion that is impregnated in said lotion-impregnated sheet includes at least one of bug spray, sunburn lotion, dry skin lotion, sunscreen, tanning lotion, pain relieving lotion, muscle relaxing lotion, acne medication, anti-itch lotion, self-tanning lotion, hair removal lotion, body hair bleaching agents, perfume, and topical medications; and
a fastener that is associated with at least one of said lotion-impregnated sheet and said applicator body and that removably attaches said lotion-impregnated sheet to said applicator body.

8. The lotion applicator of claim 7 wherein said fastener includes a flexible member that is received in a hole in said applicator body and that includes at least two radially inwardly projecting, flexible members that engage said lotion-impregnated sheet.

9. The lotion applicator of claim 7 wherein said handle and said applicator body are integrated.

10. The lotion applicator of claim 7 wherein said handle includes a first connector and said applicator body includes an end portion with a second connector and wherein said first connector mates with said second connector to attach said handle to said applicator body.

11. The lotion applicator of claim 7 wherein said applicator body includes a top surface having an arcuate cross section and a bottom surface having a substantially planar surface.
